(19) United States
(12) Patent Application Publication
(10) Pub. No.: US 2021/0100230 A1
(43) Pub. Date: Apr. 8, 2021

(54) SYSTEMS AND METHODS FOR AUTOMATED AQUATIC INSECT REARING

(71) Applicant: Verily Life Sciences LLC, South San Francisco, CA (US)

(72) Inventors: Matthew Metlitz, Menlo Park, CA (US); Peter Massaro, San Carlos, CA (US); Victor Criswell, Burlingame, CA (US); Bradley White, San Francisco, CA (US); Angus Kitchell, San Francisco, CA (US); Johanna Ohm, Pacifica, CA (US)

(73) Assignee: Verily Life Sciences LLC, South San Francisco, CA (US)

(21) Appl. No.: 16/593,210

(22) Filed: Oct. 4, 2019

(51) Int. Cl. A01K 67/033; A01K 29/00

(52) U.S. Cl. CPC A01K 67/033; A01K 2227/706; A01K 29/005

(57) ABSTRACT

An example system for automated aquatic insect rearing includes a frame; at least one trough having a drain, the trough supported by the frame; a drain valve coupled to the drain; a lid coupleable to the at least one trough to cover the trough, the lid defining at least one opening; a fluid supply tube connectable to a fluid supply, the fluid supply tube routed to provide fluid to the at least one trough; a fill valve coupled to the fluid supply tube, the fill valve positioned and configured to regulate a flow of fluid into the at least one trough; a fill pump coupled to the at least one fluid supply tube and coupleable to the fluid supply to pump fluid from the fluid supply through the at least one supply tube; at least one feeding mechanism to dispense food into the at least one trough; and at least one fluid level sensor positioned to detect a fluid level within the at least one trough.

### FIELD

The present application generally relates to insect breeding and more particularly relates to systems and methods for automated aquatic insect rearing.

### BACKGROUND

The mass-rearing of insect larvae can be very labor intensive. A lab technician may manually add a number of eggs or insect larvae to a plastic tray and determine the amount of food and water to add into the trough for the insect larvae. The lab technician may hand carry the plastic trough to a storage area to store the plastic trough. Periodically, the lab technician may perform observations on the insect larvae in the plastic trough or add food and water as needed, or manually transfer the insects to another location once they have reached a desired level of maturity.

### SUMMARY

Various examples are described for systems and methods for automated aquatic insect rearing. One example system includes a frame; at least one trough having a drain, the trough supported by the frame; a drain valve coupled to the drain; a lid coupleable to the at least one trough to cover the trough, the lid defining at least one opening; a fluid supply tube connectable to a fluid supply, the fluid supply tube routed to provide fluid to the at least one trough; a fill valve coupled to the fluid supply tube, the fill valve positioned and configured to regulate a flow of fluid into the at least one trough; a fill pump coupled to the at least one fluid supply tube and coupleable to the fluid supply to pump fluid from the fluid supply through the at least one supply tube; at least one feeding mechanism to dispense food into the at least one trough; and at least one fluid level sensor positioned to detect a fluid level within the at least one trough.

One example method includes dispensing a quantity of water into a trough of an automated insect rearing system, the automated insect rearing system comprising: a frame, at least one trough having a drain, the trough supported by the frame, a drain valve coupled to the drain, a lid couplable to the at least one trough to cover the trough, the lid defining at least one vent, a fluid supply tube connected to a fluid supply, the fluid supply tube routed to provide fluid to the at least one trough, a fill valve coupled to the fluid supply tube, the fill valve positioned and configured to regulate a flow of fluid into the at least one trough, a fill pump coupled to the at least one fluid supply tube and coupled to the fluid supply to pump fluid from the fluid supply through the at least one supply tube, at least one feeding mechanism to dispense food into the at least one trough, and at least one fluid level sensor positioned to detect a fluid level within the at least one trough; dispensing a quantity of insect larvae into the trough; dispensing a quantity of food into the trough; coupling a lid to the trough, the lid defining an opening; activating, by the computing device, the fill pump to pump water from the fluid source into the fluid supply tube; activating, by the computing device, an actuator to tilt the trough to distribute the food and larvae within the trough; and activating, by the computing device, the actuator to return the trough to a substantially level position.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 shows a block diagram of a system 100 for automated aquatic insect rearing;

FIG. 2 shows an example system for automated aquatic insect rearing;

FIG. 3 shows a partial three-dimensional view of the interior of a frame of a system for automated aquatic insect rearing;

FIG. 4 shows an example aquatic rearing trough suitable for use with various systems and methods for automated aquatic insect rearing according to this disclosure;

FIG. 5 shows another example trough for automated aquatic insect rearing;

FIGS. 6A-6B depict a diagram of a trough sitting atop a heating element and a trough support within a frame;

FIGS. 7A-7B illustrate an example tilting mechanism;

FIG. 8 shows a cross-sectional view of an example system for automated aquatic insect rearing;

FIGS. 9-13 illustrate an example system for automated aquatic insect rearing;

FIG. 14 shows an example method for automated aquatic insect rearing; and

FIG. 15 shows an example computing device suitable for use in example systems or methods for automated aquatic insect rearing according to this disclosure.

### DETAILED DESCRIPTION

Examples are described herein in the context of systems and methods for automated aquatic insect rearing. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

Rearing insects in large quantities requires careful attention to and regulation of the rearing environment. For example, over the course of a week, a population of mosquito larvae may mature into pupae in a mass-rearing environment. During this time, the mosquito larvae need to be fed and kept in still water at a suitable, stable temperature. However, during this time, metabolic waste products build up in the water that, combined with any uneaten food or other organic debris, will tend to foul the water, potentially harming or killing some of the larvae. Thus, the number of larvae that can be successfully reared in a given container is limited based in part on the rate at which these materials foul the water. In a mass rearing environment, maximizing yield is of high importance and existing systems reach a limit of larvae per unit volume of a trough due to these constraints. In addition to yield, the cost per mosquito larvae needs to be minimized to ensure a rearing program does not terminate early or overrun budgets due to excessive cost. Because mosquito rearing programs are very manually intensive programs, these costs tend to be spent on people who tend to the mosquitos during development.

To reduce the cost of large-scale insect rearing, while also increasing population density within a particular trough, an example system according to this disclosure employs a computer controlled, closed-loop control system to maintain a stable aquatic rearing environment for insects. The example system employs a number of troughs in the form of troughs approximately three feet long by six feet wide, with a depth of approximately half an inch to two inches. Each trough is equipped with two drains, a temperature sensor, and a level sensor, and is covered by a lid that has a small vent, approximately one to five square inches in size, which allows air to move into and out of the trough and may be employed to dispense food into the trough.

The troughs may be equipped with other sensors as well, such as water quality sensors, e.g., ammonia sensors or water clarity or turbidity sensors. In addition, a peristaltic water pump is connected to the trough via one of the two drains. The second drain is regulated by an electrically actuatable ball valve. The peristaltic pump can be used to slowly drain a precise quantity of water from the trough to cycle out fouled water, which can then be replaced with fresh water as will be discussed below. The ball valve can be opened to entirely drain the contents of the trough, such as to remove the matured insect population and prepare the trough for a new population of immature insects. In addition, the troughs are equipped with tubing to enable fresh water to be added to each trough as needed (and discussed in more detail below).

In operation, a computer system is connected to each of the sensors to receive the sensor input and is also connected to the peristaltic pump and the ball valve so that each may be controlled by the computer system rather than manually operated by a person.

The troughs are placed within a frame structure that provides a physical support for each trough. The frame itself provides multiple locations at which troughs may be positioned. It is enclosed by an set of plenum walls having a number of holes formed in them to enable movement of air into and out of the frame. These plenum walls are further enclosed by solid walls, thereby creating a gap between the plenum walls and the solid walls. Heated (or cooled) air may be blown into the gap between the walls on the sides of the frame where it passes through the plenum walls into the interior of the frame, where the troughs are positioned. Air within the frame then is drawn into a climate control system, where it can be recycled or vented into the atmosphere. The computer system above may receive ambient air temperature information and control the climate control system to maintain a preset temperature within the interior of the frame.

Within the frame, each trough will rest on a discrete supporting shelf on top of a heating pad. In use, each trough will be filled to a predetermined level with clean water and a population of mosquito eggs or larvae will be deposited into the water. The heating pad is used to maintain a substantially constant water temperature, while the system also includes a climate control system and one or more ambient air temperature sensors to regulate the temperature of the climate within the frame, as discussed above. The computer system above may receive water temperature information for each individual trough and control the corresponding heating pads to maintain a preset temperature within the interior of the frame.

In this example, the frame structure stores the troughs in a grid arrangement of six vertical columns, where each vertical columns corresponds to a population of insect larvae deposited into the troughs of that column on a particular day. For example, on day 1, the troughs of column 1 may have a new population of insect larvae added to it. On day 2, a new population of insect larvae may be added to the troughs of column 2, and so forth. Thus, larvae within a particular column of troughs will reach the pupae stage on approximately the same day. The population then may be removed, the troughs cleaned, and a new population added to restart the rearing cycle. Thus, in steady state operation, a new larvae population will mature each day, which may provide a steady flow of insects through a rearing program.

To maintain a healthy larvae population, the aquatic rearing system includes a reservoir of clean water, maintained at a constant temperature within 2-3 degrees of the water temperature within the troughs, and tubing that runs through the frame past each of the troughs. To simplify the plumbing, a dedicated manifold is used for each vertical column of troughs to distribute water to individual supply tubes for each trough in the respective column, and a further manifold is used to distribute water from a single outlet on the reservoir to each of the dedicated manifolds. During operation, a pump continuously circulates water through the tubing and back into the reservoir. At each trough, an actuatable ball valve is used to allow fresh water to be added, e.g., by dripping, into a trough when needed. In this example, the computing system receives water level information from the water level sensor and actuates the ball valve to add water when the water level drops below a threshold and closes the ball valve once a second threshold level has been reached.

In addition to supplying fresh water, the system employs the peristaltic drain pumps to drain dirty water or debris from the troughs. To do so, the computer system may operate the peristaltic pump to pump out a metered amount of water, or at a specific flow rate. The level sensor may detect the reduced volume of water in the system and provide signals to the computer system, as discussed above, indicating a low water level below a threshold. The computer system may then actuate a ball valve to supply fresh replacement water to the trough. The troughs may also (or instead) be equipped with an additional peristaltic pump that can add a metered amount of fresh water at substantially the same rate as another peristaltic pump drains waste water from the trough. The computer system may operate the peristaltic pumps continuously, on a preset scheduled, or based on sensor signals received from one or more water quality sensors.

While the system can adjust water quality using the fresh water supply and the drain pump, it can also gently disturb the water surface to breakup any film or scum. This example system includes air hoses plumbed to a pressurized air source using manifolds, generally as described above with respect to the fresh water distribution system. The air hoses terminate in a respective trough, where they may be curved or coiled on the bottom of the troughs to provide full coverage of the trough. The portions of the air hoses within the troughs have holes formed in them to allow pressurized air to escape as bubbles, which rise to the surface of the water, disrupting material on the surface. The ends of the tubes may be plugged to help reduce the overall air flow needed within the air tubing.

This example aquatic rearing system includes feeding mechanisms connected to a food source that can then distribute food to each of the troughs. This example system employs a robotic arm to maneuver the feeding mechanism into place over the vent in a trough's lid where food can be dispensed into the container. However, some examples may include a feeding mechanism, e.g., an augur, per trough. The computing system activates the feeding mechanism, whether the robotic arm or individual feeding mechanisms, according to a predetermined feeding schedule. In the case of the robotic arm, the computer may specify individual troughs into which to dispense food. In examples where each trough (or each column of troughs) has a dedicated feeding mechanism, the computer may individual control each feeding mechanism to dispense food into the respective troughs.

To help distribute larvae and food within the troughs, the system also employs actuator mechanisms to tilt the troughs by raising the side of the troughs opposite the drain. In this example, a rotary motor with a cam is positioned below the lowest trough in each vertical column. The rod has projections extending under a side of each trough. To tilt the containers, the computer system actuates the rotary motor, which turns the cam until a cam lobe vertically displaces the rod, thereby raising the corresponding side of each trough. This may help distribute the food or the larvae more evenly within the troughs. The motor may then rotate the cam again to return the troughs to a level resting position. This action may be repeated as needed to distribute the larvae or food within the troughs, such as on a fixed schedule.

Once the larvae within a vertical column of troughs has reached a desired developmental stage, the troughs may be drained in-place using the drains and the tilting mechanism. In particular, the drains of each of the troughs in a column are connected to a common collection basin by one or more drain tubes. To drain a trough, the system actuates the tilting mechanism to tilt the troughs in the column and then opens the drain valves. The contents of the rearing mechanism may then drain under the force of gravity through the open drain valve into the collection basin. However, in some cases, additional clean water may be pumped into the trough to help force the trough's contents to drain or the lid vent may be closed and pressurized air may be pumped into the trough to force the contents to drain through the drain valve.

After the containers have been drained, the troughs are cleaned. In this example, a robotic arm maneuvers into each trough and sprays water or spins a brush across the interior of the trough to flush any remaining contents out through the drain. At this time, the collection basin with the collected insects has been removed and the cleaning water may then drain into a waste collection basin or into a buildings sewer piping. Once the troughs have been cleaned, they may then be refilled with water and another population of insect larvae.

The example system described above provides significant advantages over traditional mass-rearing systems that employ manual labor to monitor insect populations, distribute food, transfer insects from troughs, and clean the troughs. In particular, the system described above employs sensors and computer control to maintain a substantially constant temperature aquatic environment for maturing insects, both by controlling heating pads for individual troughs and by regulating an ambient air heater and blower system to blow conditioned air through the plenum wall into the interior of the frame, and recycle (or vent) air from the interior of the frame.

In addition, the system autonomously maintains water quality by cycling out dirty water while adding fresh water. The computer can control the rate at which water is drained by operating the drain pump at a particular flow rate, such as based on a sensed water quality, while also adjusting the inflow of fresh water by actuating a valve on the fresh water inlet tubing based on a deviation from a desired water level. By maintaining consistent water quality the system enables higher densities of mosquito larvae to be reared in a container of any particular size.

Further, the system enables mixing of the contents within the troughs by providing computer-controlled actuators to tilt the troughs. Such action can help evenly spread food or larvae within the troughs, both helping to spread out the larvae to help reduce overcrowded regions, and to help ensure food is distributed to the entire larvae population. These actuators can also be used to drain the contents of the troughs once the insects have reached a desired level of maturity.

By implementing the features discussed above or others discussed throughout this disclosure, insect rearing can be accomplished with more consistent results, at higher densities, and with higher yields that conventional insect rearing systems. Further, example systems and methods can significantly reduce the cost of mass-rearing populations of insects.

This illustrative example is given to introduce the reader to the general subject matter discussed herein and the disclosure is not limited to this example. The following sections describe various additional non-limiting examples of systems and methods for automated aquatic insect rearing. It should be appreciated that “aquatic insect rearing” refers to rearing insects that have a lifecycle that includes an aquatic stage and is not limited to insects that are aquatic insects as adults. For example, examples according to this disclosure may be used to rear insects such as Aedes aegypti and Aedes albopictus mosquitoes.

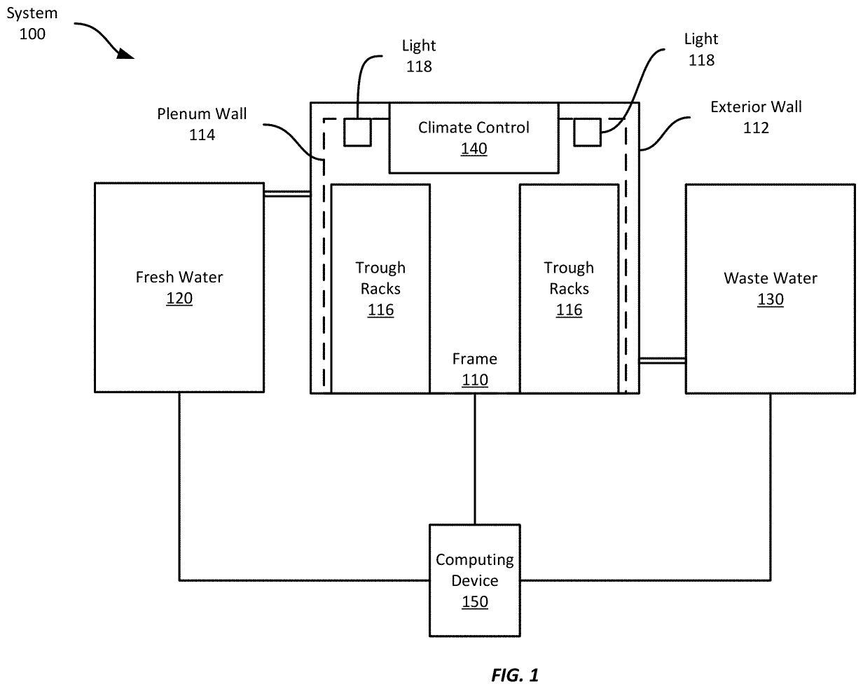

Referring now to FIG. 1, FIG. 1 shows a block diagram of a system 100 for automated aquatic insect rearing. The system 100 includes a frame 110, a fresh water supply 120, a waste water system 130, and a computing device 150 to receive sensor signals, and control different portions of the system 100. The frame 110 includes an exterior wall 112, a plenum wall 114 that define an interior room in which multiple trough racks 116 are positioned, and one or more lights 118 that may be used to provide lighting within the system 100 or to provide day/night cycles to the insect populations. Each of the trough racks 116 can support one or more columns of troughs in which insects may be aquatically reared. The frame 110 also includes a climate control system 140, such as a heating, ventilation, and air conditioning (“HVAC”) system, humidity control, etc. Within the frame, the climate control system 140 recirculates air by drawing in air, adjusting its temperature or humidity, and expelling it back into the frame 110 to maintain predetermined environmental conditions, e.g., temperature and humidity.

The exterior wall of the frame is solid and non-porous, such as a metal, glass, or other suitable material. In some examples, the frame may be constructed within a standard ISO shipping container, with the walls of the container operating as the exterior wall 112. The plenum wall 114 is constructed of any suitable material, such as Lexan, wood, aluminum, steel, etc., and defines openings through which air may circulate. In addition, the plenum wall 114 is positioned to define an air gap between the plenum wall 114 and the exterior wall 112. As will be discussed in greater detail below, the climate control system 140 can blow air into the air gap between the two walls 112, 114, which then passes through the plenum and the trough racks, and into the interior of the frame, where it is then returned to the climate control system for further processing and recirculation.

The trough racks 116 define multiple positions on which aquatic rearing troughs may be positioned. The positions are arranged in one or more vertical columns running the length of the frame. Each column can support one or more aquatic rearing troughs, with sufficient space between troughs to enable free movement of air across the troughs, distribution of food into the troughs, and movement of the troughs, such as tilting, to enable mixing or draining of trough contents. It should be appreciated that any suitable arrangement of troughs may be used, however.

The fresh water supply 120 may be any suitable system that can store and provide fresh water. In general, the fresh water supply 120 includes one or more reservoirs capable of holding a quantity of water, and one or more pumps to pump water from the reservoir(s) through tubing into the frame 110, where it may be distributed by a plumbing system, including tubing, valves, and in some examples, additional pumps, to aquatic rearing troughs. In some examples, the fresh water system may include a reverse osmosis system to filter water from another water supply, e.g., a public water supply. In some examples, the fresh water supply may include systems to treat or filter water recirculated from the waste water system 130. It should be appreciated that “fresh” water denotes water that is sufficiently clean such that it can be used to rear insect eggs, larvae, or pupae. For example, the water need not be potable water, but should have concentrations of dissolved metals, salts, ammonia, etc. below toxicity thresholds for the insects to be aquatically reared.

The waste water system 130 is any suitable system that can receive waste water from the troughs within the trough racks 116 and process it or dispose of it, such as by draining into a public sewage system. In this example, the waste water reservoir includes a biosump system to process the waste water. The biosump system includes mechanical filters, biofilters, UV filters, protein skimmers, and ozone filters clean the water to a level with an acceptable amount of beneficial bacteria such that the water can be recycled and used to rear additional insect populations. Such a biosump can provide biological inertia and prevent significant unwanted changes in water quality, which are thereby less likely to shock the aquatic rearing environments. In addition, water from another source, such as a reverse osmosis system, or even a public water supply, can be injected into this system to aid with process control and system turnover.

Other water conditioning systems with lesser or different features than the biosump system discussed above may be employed, such as by using mechanical filters (e.g., skimmers), UV light, protein skimmers, ozone filtration, etc. Further, beneficial bacteria may be introduced into the treated water, or at the “fresh” water supply before the water is introduced into the aquatic rearing environments to provide health and environmental additional benefits to the insect populations.

In some examples, the waste water system 130 may also receive matured insects from troughs within the trough racks. For example, when the insects reach a desired level of maturity, or have spent a preset period of time within a trough, they may be drained, along with the contents of the trough, into the waste water system, where they are mechanically filtered into a container. The container may then be retrieved or moved, e.g., along a conveyor, to another station where the matured insects may be retrieved. For example, the insects may be drained onto a strainer, which allows water to pass through while collecting the insects. The contents of the strainer may then be emptied into a container.

One or more sensors may be used to detect water quality, such as pH, dissolved oxygen, alkalinity, total dissolved solids (TDS), hardness, iron, magnesium, calcium, manganese, aluminum, conductivity, sodium, fluoride, potassium, copper, sulfate, phosphorus, ammonia, nitrate, nitrite, dissolved organic carbon (DOC), dissolved organic matter (DOM), oxidation-reduction potential (ORP), hydrogen sulfide (H2S), temperature, etc. Such sensors may be used to adjust water processing components to maintain a predetermined concentration or level of such materials.

The computing device 150 receives sensor information from various sensors, such as within the frame 110, the troughs, or the waste water system 130, and can control different systems based on the sensor readings. In addition, the computing device 150 may control other systems based on other inputs, such as time or user commands. In this example, the computing device 150 receives climate information from one or more temperature sensors or humidity sensors within the frame 110. The computing device 150 can then adjust the output of the climate control system 140 based on the received climate information. For example, the computing system 150 may be instructed to maintain a temperature of approximately 82 degrees and 60% humidity, and based on the sensed climate information, may adjust the temperature of the climate control system 140 or the amount of moisture in the air to maintain the preset temperature and humidity.

In addition, the computing device 150 may receive temperature information, water level information, water quality information, etc. from one or more sensors disposed at each trough position within the trough racks 116. For example, troughs positioned within the racks 116 may include water level sensors or water quality sensors. The computing device 150 may receive sensor information from such sensors and determine whether additional fresh water should be added to the respective trough or whether waste water should be drained from the trough. To do so, the computing device 150 may transmit a signal to an electronically actuatable valve at the water inlet to add fresh water or to a drain pump to drain waste water, or to adjust the rate at which water is added or drained from the respective trough. For example, the system 100 may operate such that water is continuously added and drained from each trough to help maintain a suitable aquatic environment. In this example, the system 100 operates in such a fashion at a rate that, on average, entirely replaces the water in each trough approximately every 3 hours.

The computing device 150 may also control the distribution of food to each trough. As will be discussed in more detail below, each trough may be equipped with a food distribution device, such as an augur, blow feeder, broadcast feeder. The computing device 150 may activate such food distribution devices based on a preset schedule. Alternatively, the computing device 105 may control a robotic arm having a suitable food distribution device and maneuver the robotic arm to distribute food to each trough according to a preset schedule.

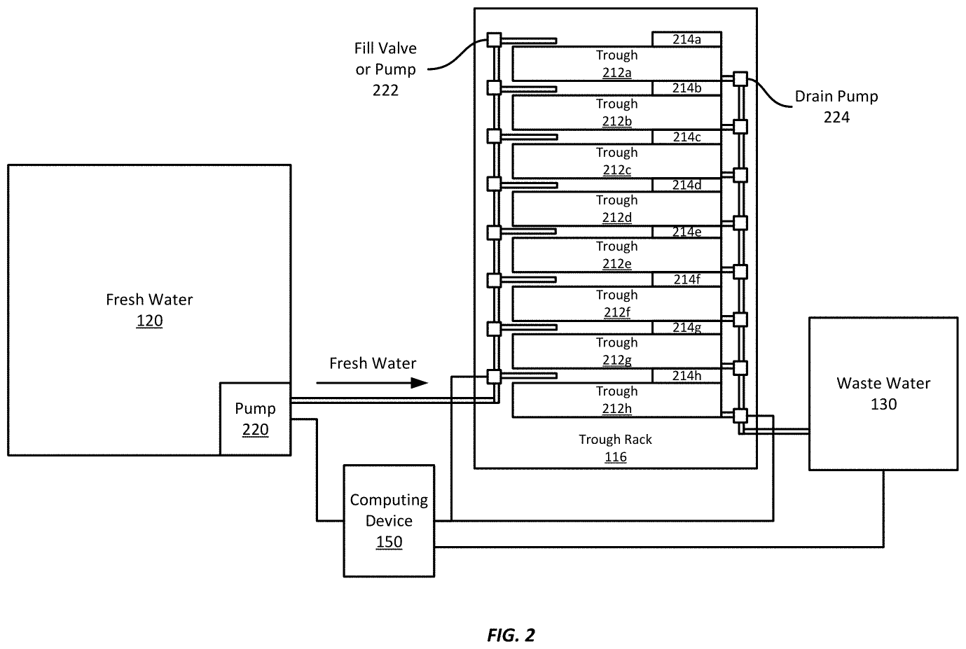

Referring now to FIG. 2, FIG. 2 shows a more detailed view of certain components of the system 100 shown in FIG. 1. As discussed above, the frame 110 stores multiple troughs 212a-h in trough racks. FIG. 2 shows one track rack 116 that supports 8 troughs 212a-h. They troughs 212a-h are stored in a vertical column and each have an associated feeding mechanism 214a-h, such as an augur feeder, blow feeder, or broadcast feeder. In addition, the troughs 212a-h are each supplied with fresh water via tubing connected to the fresh water supply 120. A pump 220 pumps water through the tubing to computer-controlled fill valves that may be opened or closed by the computing device 150 to maintain a suitable fluid level within each trough 212a-h. As is shown, each trough 212a-h. has a corresponding fill valve (or pump, e.g., peristaltic pump) 222, e.g., an electronically actuatable ball valve, meaning that the computing device 150 may individual control the fluid level in each trough 212a-h.

In addition, each trough 212a-h in the trough rack 116 has a corresponding drain pump 224, which is controlled by the computing device 150 to drain fluid out of the respective trough 212a-h. In this example, the drain pumps 224 are each peristaltic pumps that are set at a constant flow rate to continuously drain each trough 212a-h. Similarly, the fill valves are partially opened to continuously add a small amount of fresh water to each trough 212a-h. In this example, water is added and drained from each trough at a rate of approximately 200 milliliters (“ml”) per minute; however, any suitable rate may be used. Further, some examples may employ any suitable pump or may not continuously add and drain fluid, but instead, may drain fluid when one or more water quality limits are reached, or periodically according to a schedule.

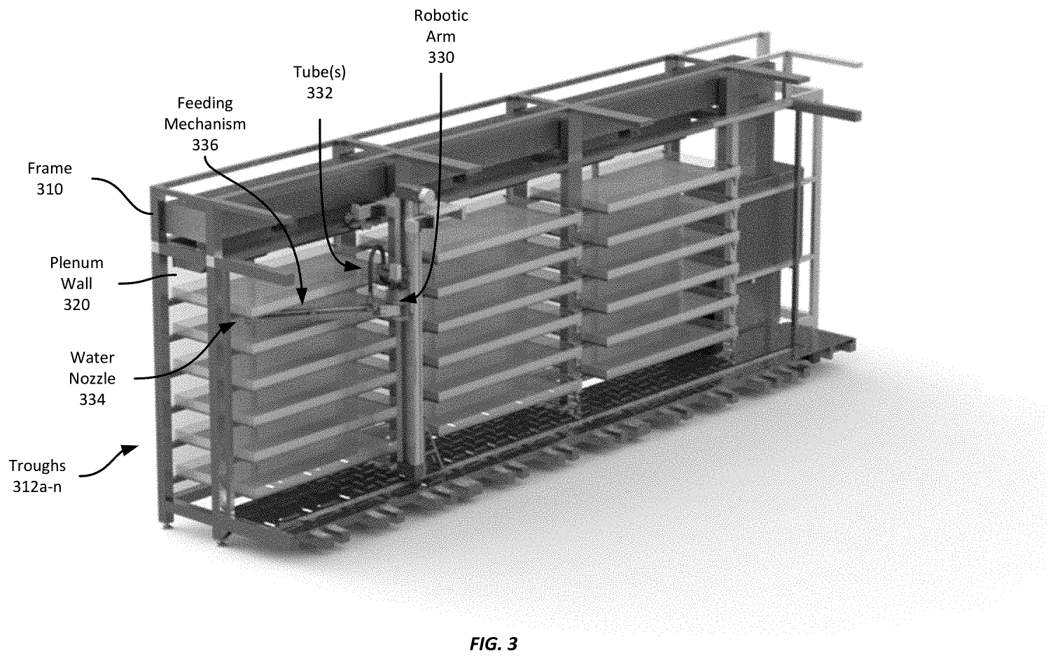

Referring now to FIG. 3, FIG. 3 shows a partial three-dimensional view of the interior of a frame of a system for automated aquatic insect rearing. As may be seen, the frame 310 includes positions to store troughs 312a-n (where “n” is any positive integer) in multiple columns. While they are not shown in this diagram, the system also includes suitable plumbing and pumps to supply fresh water to each trough, and to drain waste water and other material from each trough.

This example employs a robotic arm 330 to perform a variety of tasks. For example, the robotic arm depicted in FIG. 3 has a feeding mechanism 336 attached to it, such as an augur, blow feeder, broadcast feeder, etc. Food is supplied to the feeding mechanism by one of the tubes 332 connected to the robotic arm. The robotic arm 330 also includes a water nozzle 334 and associated tubing 332 connected to a fresh water source. In this example, the water nozzle may be used to clean a trough after it has been drained of its contents, though in some examples, the water nozzle 334 may also be employed to add fresh water to a trough 312a-n with an existing population of insects. In some examples, the robotic arm 330 may also include a dispenser to dispense immature insects, e.g., eggs or larvae, into a trough. The dispenser may be connected to tubing 332 which is connected to a container having a quantity of such immature insects.

A computing device (not shown) controls the robotic arm and the feeding mechanism and can maneuver the robotic arm into position over any trough 312 a-n in the frame 310 to automatically dispense food into the respective trough 312a-n.

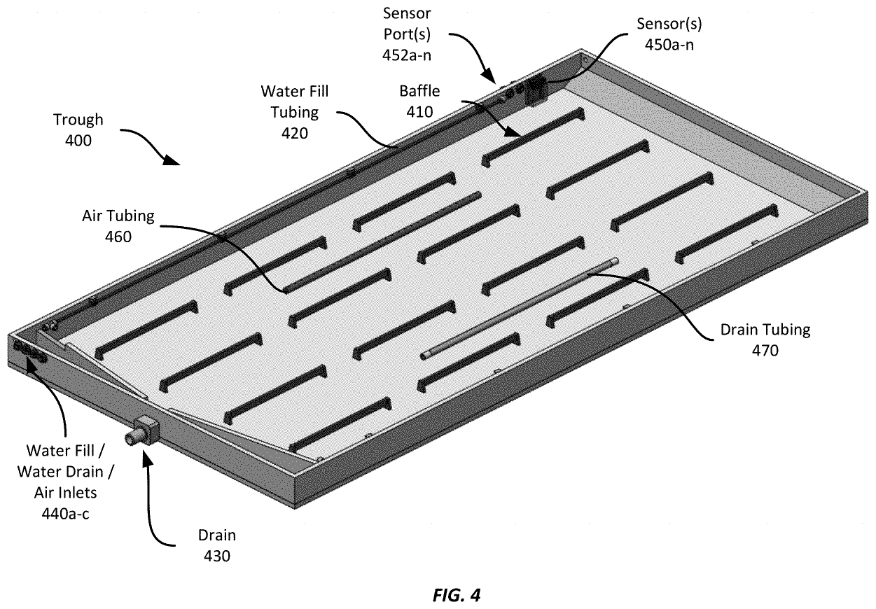

Referring now to FIG. 4, FIG. 4 shows an example aquatic rearing trough 400 (or simply trough 400), suitable for use with various systems and methods for automated aquatic insect rearing according to this disclosure. The trough 400 includes a base and sidewalls that enclose the trough volume, in which fluids and immature insects, e.g., eggs, larvae, or pupae, may be deposited and reared. This trough has approximate dimensions of 3 feet by 6 feet by 2 inches (width×length/depth×height); however, any suitable size may be employed. This example trough 400 is intended to be positioned within a frame for the lifetime of the trough 400. Thus, the trough may be filled, maintained, drained, and cleaned in-place within the frame. To accommodate this goal, the trough and frame are designed with certain features. For example, as discussed above, the insect population and other contents of the trough may be drained into a waste water system where the insect population is mechanically filtered from the remaining contents of the trough. To accommodate this, the trough's drain may be connected to tubing that runs to the filter from the troughs position within the frame. Still other features to be discussed below may further enable the trough to remain in place for its lifetime, thereby substantially reducing human labor that might otherwise be needed to fill, maintain, drain, and clean the trough 400 during an insect rearing cycle.

The trough 400 includes multiple ports defined in one or more of the sidewalls to accommodate fluid or electronic connections. In this example, the trough 400 has a flat bottom and the sidewalls are of equal height, thus the trough 400 does not have any defined slope when resting on a level surface. However, other troughs may have sloped bottoms, e.g., towards the drain port 430 to assist in draining the trough. Further, while the trough 400 has a rectangular shape in this example, any suitable trough shape may be used. For example, some troughs may get narrower from rear to front, which may help drain the troughs of fluid or insects. Further, the bottom of the trough 400 may define one or more gutters, in some examples, into which insect eggs may be deposited for hatching.

The ports for this example trough 400 include a drain 430 and water fill, water drain, and air inlets 440a-c positioned at the front side of the trough 400. The front of the trough 400 generally refers to the sidewall of the trough 400 includes the drain port 430; however, the trough may be oriented in any suitable way within a system. The trough 400 also includes water fill tubing 420 to receive water from the water fill inlet and dispense it into the trough 400.

The inlets and drain 440a-c enable fluid or air connections to external tubing, such as to provide fresh water or pressurized air, or to drain waste water from the trough. The trough in this example includes a peristaltic pump (not shown) connected to a drain 440a to allow waste water to be continuously or periodically drained from the trough 400 in highly controlled amounts. For example, a computing device may activate the peristaltic pump to run at a predetermined flow rate to drain waste water when one or more water quality sensors indicate water quality drops below a threshold. Such a peristaltic pump may be periodically back-flushed to prevent clogging. Alternatively, water may be continuously drained, with a corresponding amount of fresh water added, to maintain a suitable rearing environment.

The trough 400 also includes air tubing 460 (partial length of tubing shown) that runs along the bottom of the trough and snakes back and forth between sidewalls in this example, though such tubing may be arranged in any suitable way according to various examples. The air tubing defines small holes to enable pressurized air received via the air inlet to escape as bubbles into the fluid within the trough. The bubbles may be used to break up surface scum or other material that may collect on the surface.

A number of baffles 410 are defined on the bottom surface of the trough 400. The baffles 410 in this examples are evenly spaced in a grid-like formation on the bottom of the trough 400 and are all oriented in the same direction; however, any suitable combination of size, shape, or orientation may be used for baffles according to different examples. The baffles 410 may help disperse insect larvae within the trough as they mature.

The trough 400 also includes one or more sensors 450a-n, or mounting positions for such sensors. Troughs 400 may be equipped with sensors to provide information to a computing device, e.g., computing device 150, such as water temperature, water level, overflow indications, water quality, etc. Suitable water quality sensors may include ammonia sensors, dissolved oxygen sensors, CO2 sensors, water turbidity or clarity sensors, etc. The sensors 450a-n may be mounted to one or more sidewalls, as depicted in FIG. 4, or may be mounted in any suitable location, including the bottom of the trough, or a trough lid as will be described in greater detail below. To enable the sensors to provide sensor information to a computing device, the trough 400 also defines one or more ports to allow sensor leads to exit the trough 400 and communicatively couple with a computing device.

While the trough 400 is depicted as having certain features, it should be appreciated that different example troughs according to this disclosure may have different combinations or numbers of the features depicted in FIG. 4. Further, example troughs may include other features than those depicted here, such as one or more pumps to add in draining the trough 400, a heating element or pad disposed within the trough or adhered or otherwise positioned on the underside of the bottom of the trough (e.g., by resting the trough on top of it), charcoal filters, a hydroponic loop to remove potentially harmful bacteria or introduce helpful bacteria, one or more cameras affixed to a sidewall of the trough to monitor an insect population, etc.

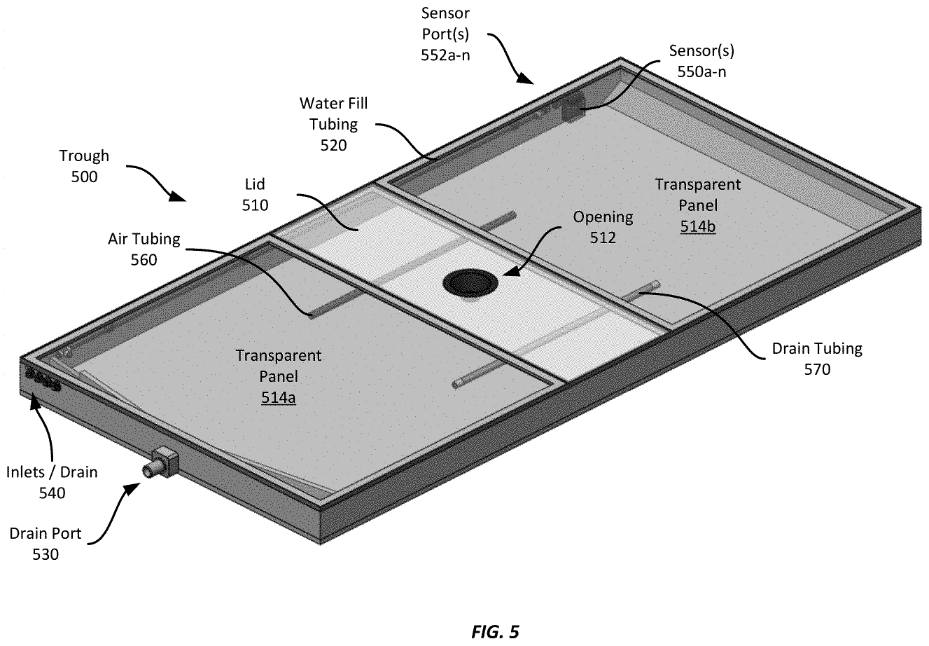

Referring now to FIG. 5, FIG. 5 shows another example trough 500 for automated aquatic insect rearing. The trough 500 in this example has substantially the same components as the trough 400 discussed above with respect to FIG. 4; however, it does not include baffles. In particular, the example trough 500 includes water fill tubing 520, one or more inlets or drains 540, a drain port 530, one or more sensor ports 552a-n and one or more sensors 550a-n, air tubing 560 (shown in part), and drain tubing 570 (shown in part).

The trough 500 also includes a lid 510, which covers the entire trough 500. The lid 510 includes two transparent panels 514a-b and a central opaque portion. The central portion of the lid defines an opening 512 through which food may be dispensed into the trough 500 and allows air to flow into and out of the trough 500. And while the lid in this example has an opaque central portion defining the opening, the entire lid 510 may be transparent and the opening 512 may be formed in the transparent lid. Further, while the opening 512 is centrally located in this example, the opening may be located in any suitable portion of the lid. Further, example lids may have multiple openings. For example, if the trough is significantly larger than the trough 500 depicted in FIG. 5, multiple openings may be used to help ensure adequate air is available and sufficient food may be deposited within the trough.

A lid 500 is provided in this example to help reduce the evaporation rate of fluid from within the trough 500. A trough without a lid may lose water at a relatively rapid rate, which may increase water usage within the system, cool the contents of the trough below a desired temperature, or increase the humidity within the aquatic rearing system above a desired level. The transparent panels in this example may be desirable to enable light to pass into the aquatic environment or to allow day/night cycles to be achieved using timed lighting within an aquatic rearing system. In this example, the lid 510 substantially prevents gas exchange except via the opening 512; however in some examples, the lid may be at least partially gas-permeable or a mesh or screen.

The lid 500 may be affixed to the trough 500 using one or more releasable coupling mechanisms, such as clips or clamps, or by a hinge or other pivot mechanism. In some examples, the lid 500 may fit within one or more grooves defined around the edge of the trough 500 or may simply rest on top of the trough 500.

Troughs similar to the trough 500 shown in FIG. 5 may be advantageous for automated aquatic insect rearing systems according to this disclosure because they may provide a more stable environment for immature insects, which may enable more accurate control over the environment by the system. As discussed above, use of a lid similar to the one depicted in FIG. 5 may reduce evaporative fluid loss and temperature loss, thereby enabling more accurate control over both fluid level and temperature. Further, the lid 510 may still allow for sufficient air exchange to provide oxygen to the aquatic environment via the opening 512.

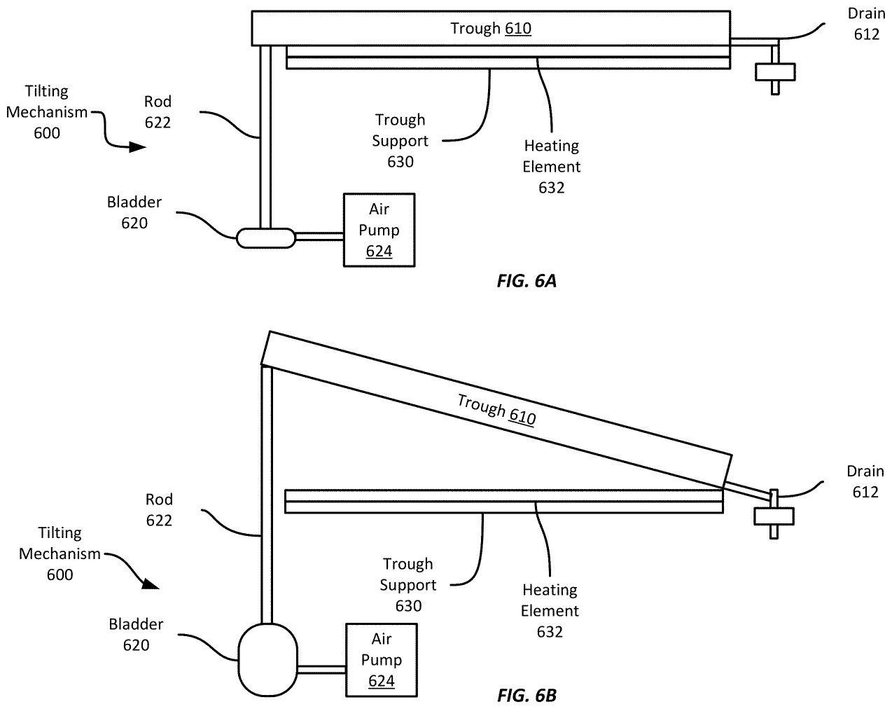

Referring now to FIGS. 6A-6B, FIGS. 6A-6B show an example tilting mechanism 600 suitable for use with example systems for automated aquatic insect rearing according to this disclosure. As discussed above, troughs to rear immature insects may be positioned in trough racks and may remain in place for their usable life spans, which typically will last through many insect rearing cycles. Further, as discussed above with respect to FIG. 5, troughs having lids may have limited openings through which food may be dispensed into the troughs. As a result, food may remain in place near the lid opening without evenly distributing throughout the aquatic environment. Similarly, metabolic waste or other contaminants may accumulate in certain regions of the trough.

To help alleviate these issues and to help distribute material, including food and immature insects, throughout the trough, or to assist the draining process for a trough, a tilting mechanism may be incorporated into some examples systems for automated aquatic insect rearing according to this disclosure.

Referring to FIG. 6A, FIG. 6A depicts a diagram of a trough 610 sitting atop a heating element 632 and a trough support 630 within a frame, such as the frame 310 depicted in FIG. 3. The front of the trough 610 has a drain outlet 612 and tubing connected to it, while a tilting mechanism 600 is positioned at the rear of the trough 610. In this example, the tilting mechanism includes an inflatable bladder 620 and a rod 622 connected to the rear of the trough 610. While the bladder is deflated, the trough 610 rests in a level, horizontal orientation. Such an orientation may be the steady-state position for the trough 610, such as while a population of immature insects is maintained in an aquatic environment.

At a later time, the bladder 620 may be inflated, such as by a computing device, e.g., computing device 150, activating an air pump 624 corresponding to the bladder 620. As the bladder 620 inflates, it pushes upward on the rod 622, which in turn raises the rear of the trough 610 as shown in FIG. 6B, tilting the trough 610. By tilting the trough 610, the contents of the trough 610 may be mixed. This mixing may be further encouraged by repeatedly inflating and deflating the bladder 620, such as over the course of a minute or a few minutes. It should be appreciated that the mixing action need not be vigorous. Rather, simply raising and lowering the rear of the trough 610 a few inches, e.g., 2-4 inches, over the course of a few seconds, e.g., 2-4 seconds, several times may mix the contents of the aquatic environment, such as by distributing the food and immature insects, or diluting concentrations of contaminants that may have accumulated in parts of the trough. Further, elevating the rear of the trough 610 may aid in draining the trough's contents when an insect rearing cycle is complete.

While the example shown in FIGS. 6A-6B elevates only a single trough 610, it should be appreciated that a column of troughs may be elevated by a single tilting mechanism. The rod 622 may have protrusions extending under each trough in the column such that when the rod 622 is raised, the rear (or other side) of each trough is also raised.

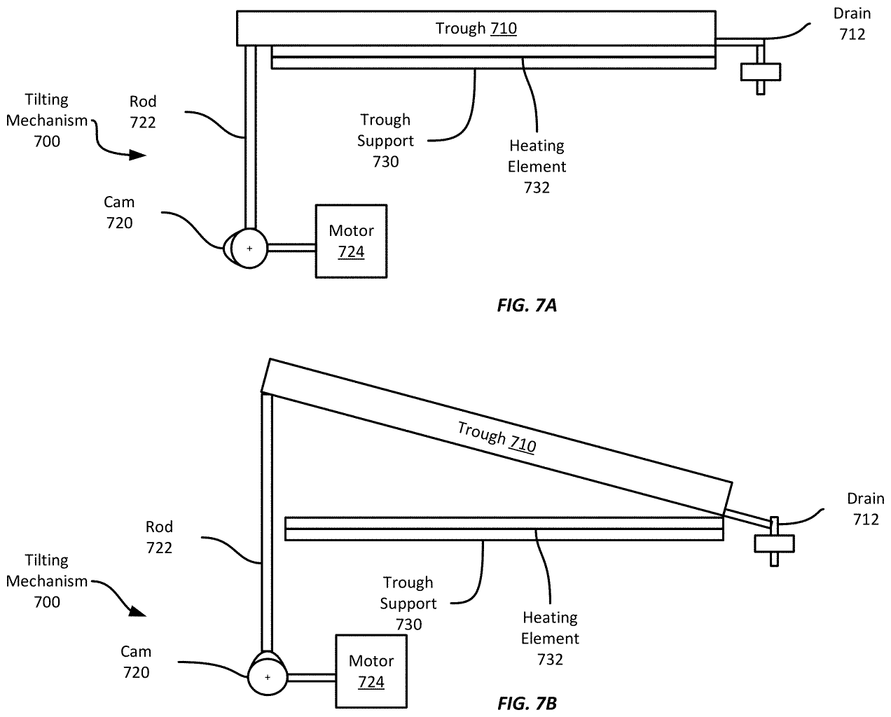

Referring now to FIGS. 7A-7B, FIGS. 7A-7B illustrate another example tilting mechanism 700. In this example, the tilting mechanism 700 includes a cam 720, a rod 722, and a motor 724. The cam 720 includes a cam lobe that rotates with the cam 720 and displaces the rod 722 when it rotates past the rod 722. The motor 724 in this example is a stepper motor that can be rotated, e.g., based on signals transmitted by a computing device, between the two positions illustrated in FIGS. 7A and 7B. As discussed above with respect to FIGS. 6A and 6B, the rear of the trough 710 may be raised and lowered over several cycles to mix the contents of the trough 710 by rotating the cam 720. Alternatively, the cam 720 can be rotated into the position shown in FIG. 7B to assist in draining the trough 710 generally as discussed above.

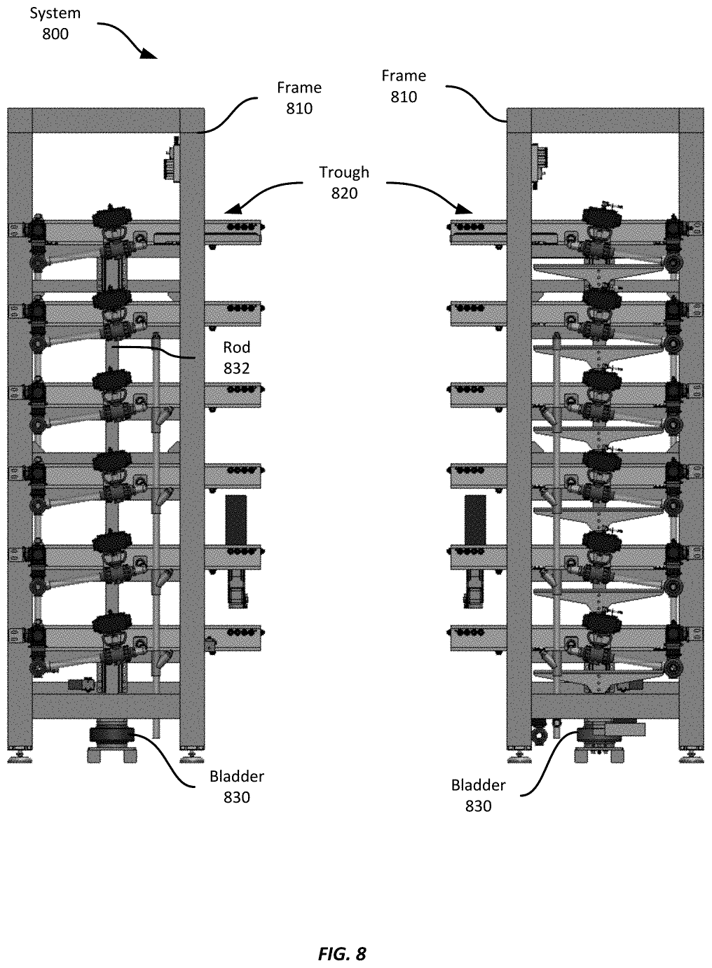

Referring now to FIG. 8, FIG. 8 shows a cross-sectional view of an example system 800 for automated aquatic insect rearing. The view shown in FIG. 8 highlights the use of a tilting mechanism for multiple troughs 820. Other aspects of the system 800 have been omitted for clarity. Illustrated in FIG. 8 are a frame 810, which supports multiple troughs 820 in multiple columns. Each column of troughs 820 has an associated tilting mechanism, each of which includes an inflatable bladder 830 and a rod 832, substantially as described above with respect to FIGS. 6A-6B. The bladder in FIG. 8 is deflated, and so the troughs 820 rest horizontally within the frame 810. However, in response to a signal from a computing device, an air pump inflates a corresponding bladder 830 to raise the corresponding rod 830, thereby tilting each of the troughs 820 in the column. It should be appreciated that each tilting mechanism may have its own air pump, or a single air pump may simultaneously inflate multiple bladders.

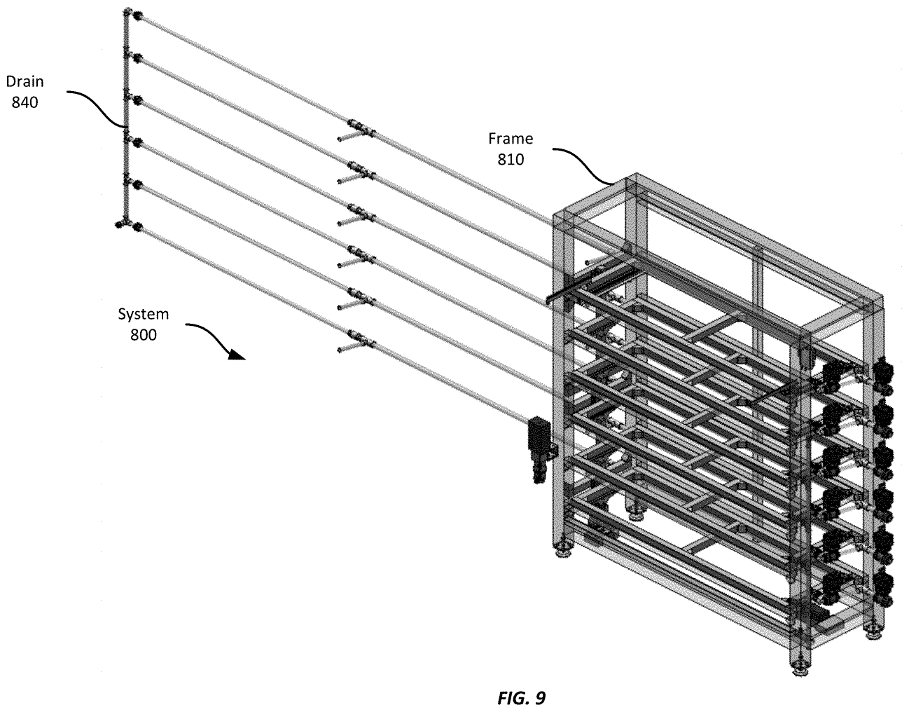

Referring now to FIG. 9, FIG. 9 illustrates another view of the example system 800 shown in FIG. 8. In this view, the drain plumbing is illustrated. As discussed above, draining waste water from individual troughs is performed to remove contaminants over time, or to entirely drain the contents of the troughs. While an insect population is maintained within the trough, a peristaltic pump may be used to continuously drain a small amount of fluid from a trough. At the same time, a computing device may monitor a fluid level within the trough based on signals received from a level sensor. As liquid is drained from the trough, the computing device may detect when the level has dropped below a threshold level, at which time, the computer may activate a pump or a valve to pump fresh water into the trough. Once the fluid level is sufficiently high, the computer may then deactivate the pump or close the valve. In this example system, the fresh water system uses hysteretic thresholds to ensure the fill system is not rapidly cycled on and off. Further, and as discussed above with respect to the tilting mechanism in FIG. 8, the troughs may also be tilted and drained entirely of their contents, including sufficiently matured insects, through the drainage system.

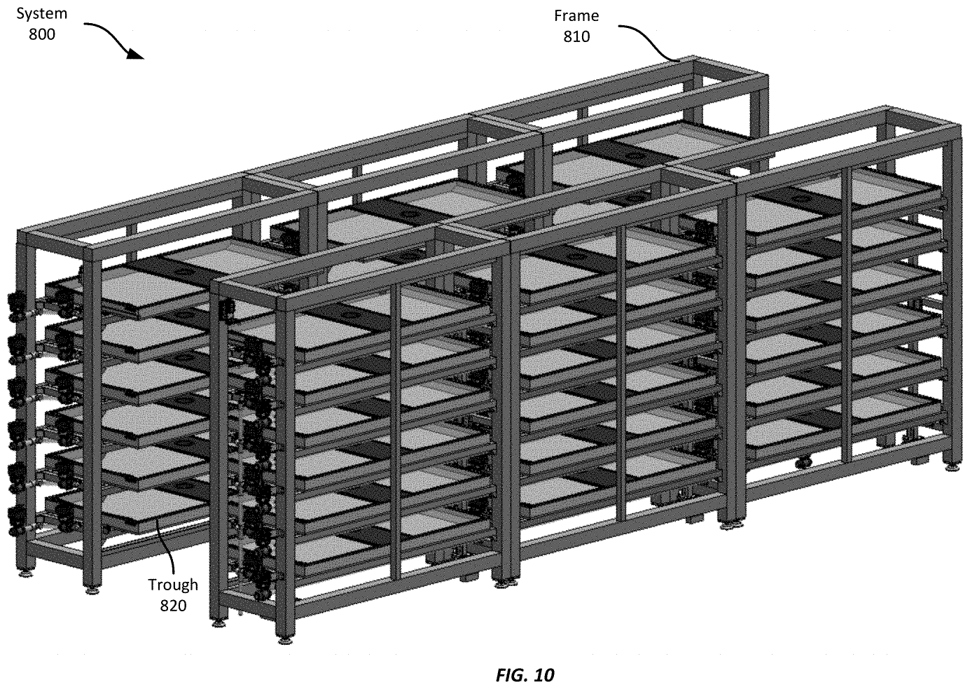

Referring now to FIG. 10, FIG. 10 shows another view of the example system 800 shown in FIG. 8. This view shows the two opposing sections of frame 810 with their respective columns of troughs. As can be seen, each frame section has three columns of troughs 820, each of which correspond to one day within a six day rearing cycle. To populate the system with immature insects, a first column is filled with water, immature insects, and food on one day. The next day, a second column is filled, and so forth, until the end of the sixth day when the first column of trays is drained and subsequently cleaned, and a new population of insects, water, and food may be added. Thus, once the trays are each populated, every days a new population of matured insects, e.g., insect pupae, is available to be drained, creating an assembly-line-style automated aquatic rearing system.

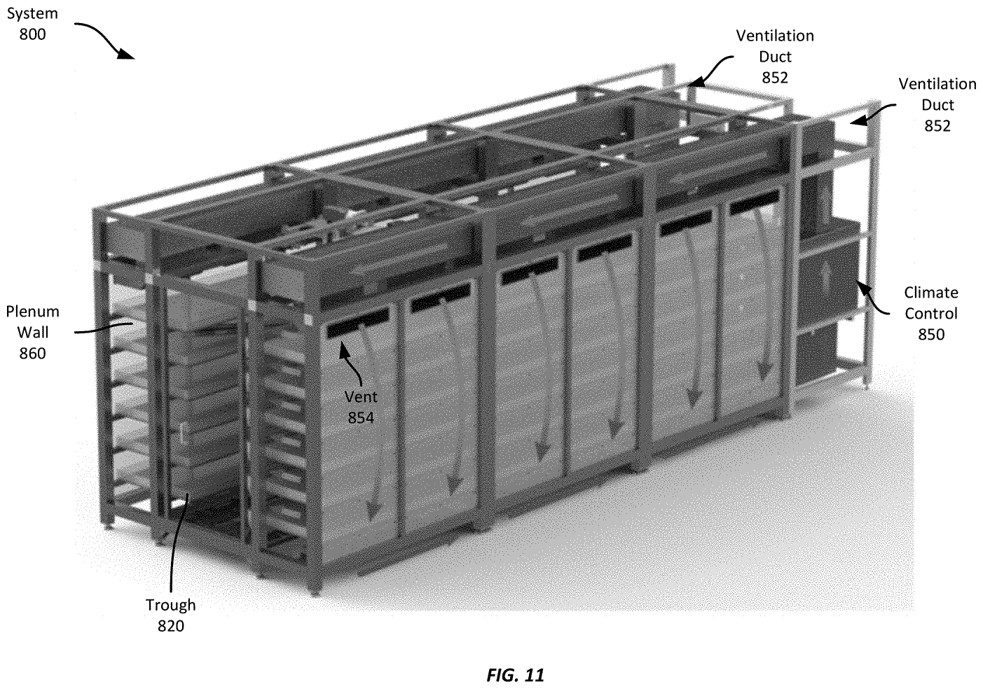

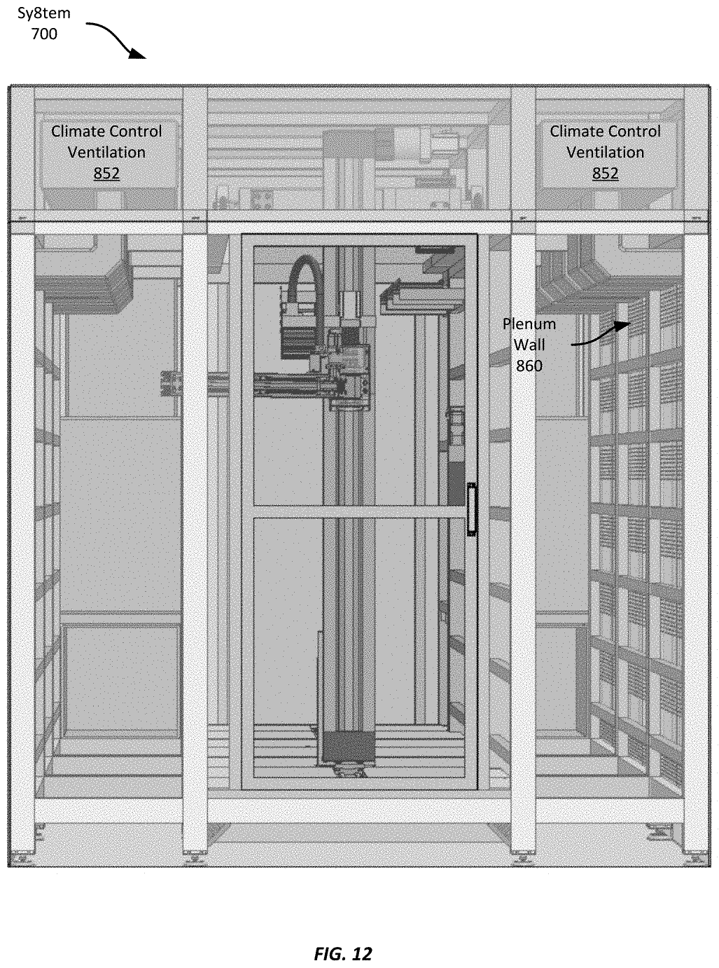

Referring now to FIG. 11, FIG. 11 shows a different view of the example system 800 shown in FIG. 8. This view illustrates the climate control system. The climate control system includes a climate control module 850, which may include an air condition unit or a heating unit, or both, depending on the rearing environment. In this example, the climate control module 850 is an air conditioning unit. Incoming air is cooled by the climate control unit and blown through ventilation ducts 852 and out through vents 854 and down into the space between the exterior wall (not shown) and the plenum wall 860. FIG. 12 illustrates a different view of the system 800 and shows the positioning of the climate control ventilation ducting and plenum wall arrangements.

The vented air travels through the plenum wall 860 and across the troughs 820, into the central walkway between the frame sections, where it can be pulled back into the climate control system 850. Thus, this system is able to recirculate air within the system to maintain a desired temperature. Further, in some examples, the climate control system 850 may also include a humidifier that can be activated when a humidity level drops below a preset level and deactivated once the humidity reaches a threshold humidity.

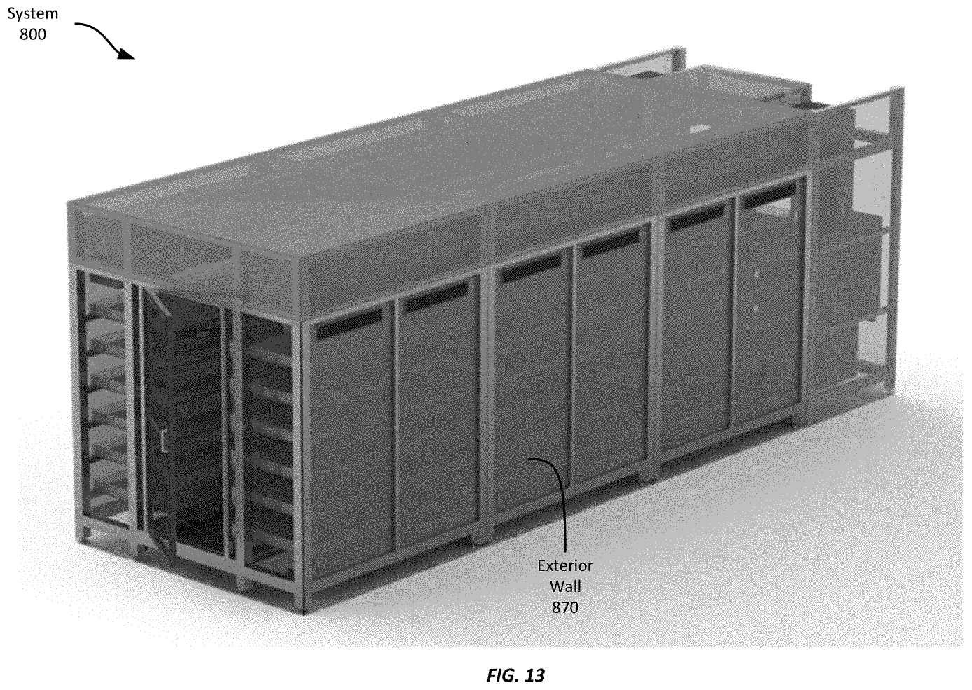

FIG. 13 shows the system 800 of FIG. 8, including the exterior wall 870, which in this example is made of a transparent material, which enables visibility into the system; however, any suitable material may be used, such as steel or aluminum. Further, the system includes a door to the system 800 to be entirely closed and also to allow personnel to enter the system and visually inspect or handle trays or other aspects of the system 800. Further, in some examples, as discussed above, the system 800 may be sized to fit within an ISO standard size shipping container (e.g., 8 feet wide by 8.5 feet tall by 40 feet long), which may enable easy transport and setup in locations where insect rearing is needed.

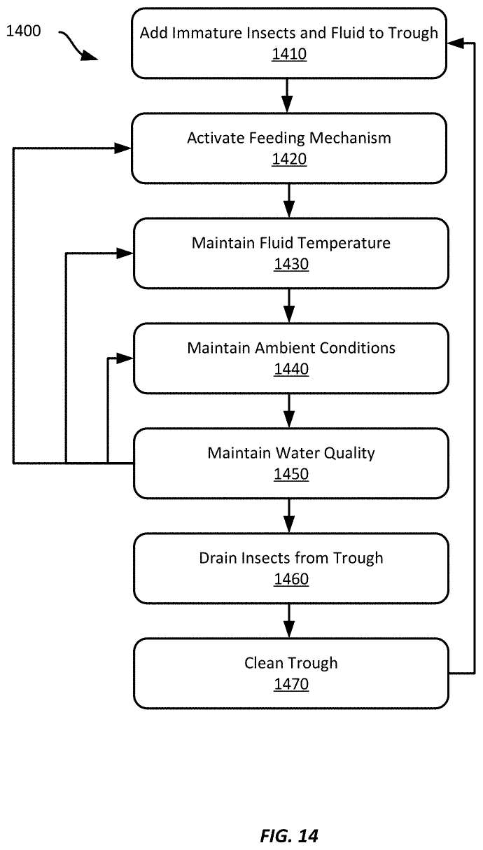

Referring now to FIG. 14, FIG. 14 shows an example method 1400 for automated aquatic insect rearing. This example method will be described with respect to the example systems shown in the figures, any suitable system according to this disclosure may be employed.

At block 1410, immature insects and fluid are added to a trough 820. In this example, immature insects and water are added to a column of troughs 820. To add the fluid, the computing device 150 activates a pump 220 connected to a fresh water source 120. The pump 220 pumps water into a pipe or tube connected to a set of water tubes running within the frame(s) 810. The computing device 150 also actuates fill valves or pumps, e.g., fill valves or pumps 222, corresponding to each trough in the column.

The computing device 150 receives sensor signals from level sensors positioned within each of the troughs within the column. Based on the sensor signals, the computing device 150 determines a fluid level for each trough and compares it against a preset fill level. When each trough's level sensor indicates that the fluid level of the respective trough has reached the preset fill level, the computing device sends a signal close the fill valve corresponding to the trough. Once each trough has been filled with sufficient water, immature insects are added.

In this example, immature insects, e.g., insect eggs or larvae, are added manually via an opening in the lids of the troughs. In some examples, however, immature insects may be added using a robotic arm. The computing device 150 may command the robotic arm to move to each trough and dispense a quantity of immature insects into the respective trough through an opening in the trough's lid. For example, the robotic arm may include one or more tubes (as seen in FIG. 3) connected to a supply of immature insects. The immature insects may be transported via a medium, e.g., water, from the source via a tube to the robotic arm, which positions the tube end over the lid opening to dispense the immature insects into each trough in the column.

It should be appreciated that while this example adds water and a new population of immature insects to a column of troughs, any suitable number of troughs may be filled at any suitable time.

At block 1420, the computing device 150 activates a feeding mechanism. In this example, the computing device 150 receives a signal indicating that a new population of immature insects has been added to one or more troughs and activates a feeding mechanism corresponding to the identified one or more troughs. Such a signal may be received in response to a user input, e.g., a manual input identifying one or more troughs into which a new population of immature insects has been added. In some examples, the computing system 150 may determine the troughs based on a predetermined schedule for each trough. An example schedule may indicate a date or time at which a trough is scheduled to be filled with a new population of immature insects. In some examples, a schedule may indicate a rotating cycle beginning with a first day on which the trough is filled with an immature insect population and ending on a last day on which the trough is drained of its contents. The schedule may then repeat indefinitely and, when the schedule indicates the trough fill day has arrived, the computing device 150 cause the trough to be filled with water and a new population of immature insects, as described above with respect to block 1410, and then activate a feeding mechanism to deposit an initial amount of food in the trough.

For feedings after the initial feeding, the computing device 150 may activate the feeding mechanism according to a predetermined feeding schedule, e.g., the system may add a predetermined quantity of food at a defined interval.

In some examples, after adding a quantity of food, the computing device 150 may activate a tilting mechanism to more evenly distribute the food within the trough(s) 820. The computing device 150 may tilt the trough one or more times in succession to mix the added food more evenly within the trough, generally as discussed above with respect to FIGS. 6A-6B and 7A-7B.

At block 1430, the computing device 150 maintains the fluid temperature within one or more troughs. In this example, the computing device 150 receives a sensor signal from one or more temperature sensors positioned within the trough(s) 820. The computing device 150 then determines the fluid temperature within the trough based on the received sensor signals and determines whether the fluid temperature is below a threshold temperature. If so, the computing device 150 activates, or increases a temperature setting on, a heating element, e.g., heating element 632. Such heating elements may be disposed within the fluid itself or may be positioned adjacent to the trough, as discussed above. The computing device 150 may then continue to monitor the fluid temperature and, when the fluid temperature exceeds a second threshold, it deactivates, or reduces a temperature setting on, the heating element.

In some examples, after heating the fluid, the computing device 150 may tilt the trough one or more times to help mix the contents and reduce any hot or cold spots within the trough. After tilting the trough, the computing device 150 may continue to maintain the fluid temperature.

It should be appreciated that the system may continuously maintain the fluid temperature within each trough, independently of other actions taken by the system, e.g., feeding, filling, draining, etc. Thus, while block 1430 is depicted within a particular sequence of blocks, it may be continuously performed.

At block 1440, the system 800 maintains an ambient conditions within the system 800. In this example, the system 800 maintains a predetermined ambient temperature and humidity within the system. In this example, the computing device 150 receives sensor signals from one or more temperature sensors and humidity sensors positioned within the system, such as at one or more locations on the frame 810, ceiling, or walls of the system 800. The temperature and humidity sensors provide sensor signals indicating a temperature of the ambient environment within the system 800. The computing device 150 receives the sensor signals and determines whether the temperature and humidity are within a target range of temperatures and humidities. If a temperature or humidity of the environment falls above an upper threshold or below a lower threshold, the computing device 150 activates a climate control system to decrease or increase, respectively, the temperature or humidity within the system 800.

For example, if the temperature sensor signals indicate the temperature exceeds an upper threshold value, the computing device 150 activates an air conditioning system within the climate control system 850 to cool the system 800. The computing device 150 then continues to monitor received temperature sensor signals to determine when the temperature falls below a second threshold, at which time, the computing device 150 deactivates the air conditioning system. Similarly, if the humidity falls below a threshold humidity level, the computing device 150 activates a humidifier within the climate control system 850 to increase the humidity level in the system. The computing device 150 then continues to monitor received humidity sensor signals to determine when the humidity rises above a second threshold, at which time, the computing device 150 deactivates the humidifier.

It should be appreciated that the computing device 850 may also have separate control over a circulation fan within the climate control system 850 and may run the fan continuously to circulate air within the system 800, or it may periodically deactivate the fan to conserve power. The computing system 150 may activate the fan on a predetermined schedule whenever the air conditioning system or humidifier is deactivated.

While this example system does not employ a heating system, in some examples, the climate control system 850 may also include, or instead include, a heating system. As with the air conditioning system, the computing device 150 may activate the heating system when temperature sensor signals indicate a temperature within the system 800 falls below a lower threshold, and then deactivate the heating system when the temperature sensor signals indicate the temperature within the system 800 rises above a second threshold.

In addition to maintaining temperature and humidity, the system 800 may also maintain certain lighting levels or a lighting schedule. In this example, the system 800 provides a simulated day/night cycle by activating and deactivating lighting within the system 800. The computing device 150 maintains a lighting schedule having simulated day and night periods. When timing information, such as a system clock, indicates a time during a simulated day period, the computing device 150 activates one or more lights within the system 800, and when the timing information indicates a time during a simulated night period, the computing device 150 deactivates one or more lights within the system 800. In some examples, the computing device 150 may slowly increase or decrease lighting levels during transitions from day to night or night to day to emulate a sunrise or sunset condition. In this example, however, the computing device 150 simply activates or deactivates lighting based on the preset day/night schedule.

Like block 1430, it should be appreciated that the system 800 may continuously maintain the temperature and humidity within the system 800, independently of other actions taken by the system 800, e.g., feeding, filling, draining, etc. Thus, while block 1440 is depicted within a particular sequence of blocks, it may be continuously performed.

At block 1450, the system maintains water quality within the troughs 810. To maintain water quality, the computing device 150 may monitor several different types of sensors for each trough, including water level, water clarity or turbidity, and the presence or concentrations of one or more substances within the environment, including carbon dioxide, ammonia, dissolved oxygen, etc. The computing device 150 receives sensor signals from each sensor installed in each trough 820. Based on the received sensor signals, the computing device 150 may activate a pump to drain fluid from one or more troughs 820 and may activate one or more valves or pumps to add additional fresh water into one or more troughs 820.

To drain waste water from a trough 820 in this example, the computing device 150 activates a peristaltic pump coupled to the respective trough. The peristaltic pump is activated to drain the fluid at a predetermined rate, e.g., 200 ml per minute. Use of a peristaltic pump may enable the computing device to drain precise amounts of fluid from the trough. In addition, the peristaltic pump may enable the computing device 150 to continuously drain fluid from the pump, while simultaneously adding fresh water to maintain an adequate amount of fluid within the trough 820. While a peristaltic pump is employed in this example, any suitable pump may be employed according to different examples. Further, some examples may not employ a drain pump, but may instead open an electronically actuatable drain valve, e.g., a ball valve, to drain fluid from the trough 820.

To add fresh water to a trough 820, the computing device 150 may activate one or more pumps coupled to the fresh water source 120. In addition, the computing device 150 may actuate one or more valves, e.g., ball valves, associated with the respective trough 820.

To determine when to add water to a trough 820, the computing device 150 receives fluid level sensor signals from one or more fluid sensors positioned within a trough 820. Based on the received fluid level sensor signals, the computing device 150 determines whether the fluid level within the trough 820 is below a first threshold level. If the fluid level is not below the first threshold level, the computing device 150 waits for another sensor signal(s). If the fluid level is below the first threshold level, the computing device 150 activates one or more fresh water pumps 220 and opens valves to allow fresh water to be pumped into the trough 820. While water is being pumped into the trough 820, the computing device 150 continues to receive fluid level sensor signals and determines when the fluid level rises above a second threshold, which is higher than the first threshold level. At this time, the computing device 150 closes the valve corresponding to the trough 820 to shut off the water supply to the trough 820. The computing device 150 may also deactivate the fresh water pump(s) 220, if no troughs 820 are in need of fresh water, which may conserve power.

When waste water is removed from a trough and fresh water is added, the computing device may also tilt the trough to help distribute the fresh water and other existing material within the trough more evenly. To do so, the computing device 150 may activate a tilting mechanism as discussed above, and may tilt the trough one or more times.

To determine when to drain water from a trough 820, assuming the system 800 is not configured to continuously drain water from the troughs 820, the computing device receives sensor signals from one or more sensors that detect various indicators of water quality. Sensors may include water turbidity sensors, water clarity sensors, ammonia sensors, carbon dioxide sensors, dissolved oxygen sensors, etc. Each sensor transmits sensor signals that are ultimately received by the computing device 150. For each sensor, the computing device 150 employs thresholds of minimum or maximum acceptable ranges. Upon exceeding a relevant threshold, the computing device 150 may drain fluid from the trough and add new fresh water to the trough as discussed above. Further, the computing device 150 may continue to replace water in the trough using such techniques until the relevant sensor signals indicate the relevant sensed characteristic as fallen below (or risen above) a relevant second threshold. If multiple different sensors indicate values beyond an acceptable range, the computing device 150 may continue to replace water within the trough 820 until all sensor signals indicate values that have passed a relevant second threshold.

Like blocks 1430 and 1440, it should be appreciated that the system 800 may continuously maintain water quality within the troughs 820, independently of other actions taken by the system 800, e.g., feeding, climate control, etc. Thus, while block 1450 is depicted within a particular sequence of blocks, it may be continuously performed.

At block 1460, the insect population is drained from the trough. In this example, the computing device 1460 determines that an insect population has been resident within a trough for a rearing cycle based on an elapsed time since the insect population was added to the trough. The computing device 150 may then open a drain on the trough and activating the tilting mechanism to tilt the trough to assist in draining the contents of the trough. The contents of the trough, including the insects pass through drainage tubing to the waste water system, where the insects are mechanically filtered from the fluid and deposited into a container for transport to another location.

At block 1470, the drained trough(s) 820 are cleaned. In this example, the computing device 150 activates a robotic arm, which positions itself to spray fresh water into the trough. The computing device 150 then uses the tilting mechanism to agitate the water within the trough 820, and then opens the drain to drain the contents into the waste water system 130. This cycle may be repeated several times to help further clean the interior of the trough 820 before the method returns to block 1410 where a new population of immature insects and fresh water are added.

It should be appreciated that each of the blocks above was described as being performed autonomously by the computing device without human intervention. However, in some examples, the computing device 150 may provide alerts indicating sensor readings that are out of acceptable or desirable ranges and suggest a response to which a human operator may trigger the corresponding action. Further, functionality described with respect to one or more blocks may be omitted from computing device control in some examples. Feeding may be accomplished manually in some examples rather than using an automated feeding mechanism and computer-based feeding schedule.

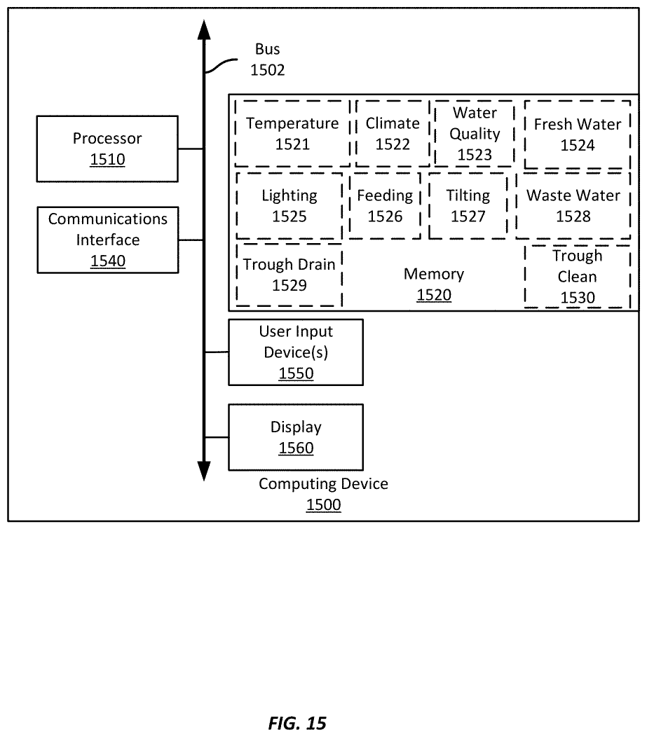

Referring now to FIG. 15, FIG. 15 shows an example computing device 1500 suitable for use in example systems or methods for automated aquatic insect rearing according to this disclosure. The example computing device 1500 includes a processor 1510 which is in communication with the memory 1520 and other components of the computing device 1500 using one or more communications buses 1502. The processor 1510 is configured to execute processor-executable instructions stored in the memory 1520 to perform one or more methods for automated aquatic insect rearing according to different examples, such as part or all of the example method 1400 described above with respect to FIG. 14. In this example, the computing device includes software to maintain temperature within one or more troughs 1521, maintain ambient climate within a system 1522, maintain water quality 1523 within one or more troughs, add fresh water 1524 to one or more troughs, control ambient lighting 1525, distribute food to one or more troughs 1526, control one or more tilting mechanisms 1527, drain or pump waste water from one or more troughs 1528, drain a trough of its contents 1529, or clean one or more troughs 1530. Examples of such functionality are described above, such as with respect to FIG. 14. The computing device, in this example, also includes one or more user input devices 1550, such as a keyboard, mouse, touchscreen, microphone, etc., to accept user input. The computing device 1500 also includes a display 1560 to provide visual output to a user.

The computing device 1500 also includes a communications interface 1540. In some examples, the communications interface 1540 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

While some examples of methods and systems herein are described in terms of software executing on various machines, the methods and systems may also be implemented as specifically-configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, that may store processor-executable instructions that, when executed by the processor, can cause the processor to perform methods according to this disclosure as carried out, or assisted, by a processor. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

That which is claimed is:

1. A system comprising:
a frame;
at least one trough having a drain, the trough supported by the frame;
a drain valve coupled to the drain;
a lid coupleable to the at least one trough to cover the trough, the lid defining at least one opening;
a fluid supply tube connectable to a fluid supply, the fluid supply tube routed to provide fluid to the at least one trough;
a fill valve coupled to the fluid supply tube, the fill valve positioned and configured to regulate a flow of fluid into the at least one trough;
a fill pump coupled to the at least one fluid supply tube and coupleable to the fluid supply to pump fluid from the fluid supply through the at least one supply tube;
at least one feeding mechanism to dispense food into the at least one trough; and
at least one fluid level sensor positioned to detect a fluid level within the at least one trough.

2. The system of claim 1, further comprising one or more computing devices in communication with the heating element, the fill pump, the at least one feed mechanism, and the at least one sensor, the one or more computing devices comprising processor executable program code to cause one or more of the computing devices to:
receive fluid level sensor signals from the at least one fluid level sensor;
determine the fluid level within the at least one trough based on the received fluid level sensor signals;
in response to determining that the fluid level is below a first threshold fluid level, transmit a signal to cause the fill valve to increase an amount of fluid provided to the at least one trough from the fluid supply tube; and
in response to determining that the fluid level is above a second threshold fluid level, transmit a signal to cause the fill valve to decrease the amount of fluid provided to the at least one trough from the fluid supply tube.

3. The system of claim 2, wherein the drain valve comprises a drain pump, and wherein the one or more computing devices further comprising processor executable program code to cause one or more of the computing devices to:
determine an elapsed time since a population of insect larvae was deposited within the at least one trough;
determine a fluid replacement rate based on the elapsed time; and
transmit a signal to establish a flow rate of the drain pump based on the fluid replacement rate.

4. The system of claim 3, wherein the drain pump comprises a peristaltic pump.

5. The system of claim 3, further comprising:
an actuator in physical communication with the at least one trough, the actuator positioned and configured to apply a force to the at least one trough to tilt the at least one trough,
a drain tube coupled to the drain valve, and
wherein the one or more computing devices further comprising processor executable program code to cause one or more of the computing devices to, in response to determining an unloading time based on the elapsed time:
transmitting an unload signal to the drain valve to open the drain valve; and
transmitting an actuator signal to the actuator to cause the actuator to tilt the at least one trough towards the drain.

6. The system of claim 2, further comprising a fluid quality sensor, and wherein the one or more computing devices further comprising processor executable program code to cause one or more of the computing devices to:
receive fluid quality sensor signals from the fluid quality sensor;
determine a fluid quality based on the received fluid quality sensor signals;
in response to determining that the fluid quality is below a first threshold fluid quality level, transmit a signal to cause the drain valve to increase an amount of fluid dispensed from the at least one trough; and
in response to determining that the fluid quality is above a second threshold fluid quality level, transmit a signal to cause the drain valve to decrease the amount of fluid dispensed from the at least one trough.

7. The system of claim 6, wherein the fluid quality sensor comprises one of an ammonia sensor, a carbon dioxide sensor, a dissolved oxygen sensor, or a light sensor.

8. The system of claim 1, wherein the at least one trough comprises a plurality of troughs, each trough having a drain, a subset of the plurality of troughs supported by the frame;
wherein the system defines a first grouping comprising:
the subset of the plurality of troughs,
a plurality of lids couplable to the troughs to cover the respective trough, each lid defining at least one vent;
a plurality of drain valves, each drain valve coupled to the drain of a different one of the troughs of the subset;
a plurality of fill valves coupled to the fluid supply tube, each fill valve corresponding to one trough of the subset;
a plurality of heating elements, each heating element of the plurality of heating elements positioned to apply thermal energy to a different one of the troughs of the subset; and
a plurality of fluid level sensors positioned to detect fluid levels, each fluid level sensor positioned to detect the fluid level of a different one of the troughs of the subset.

9. The system of claim 8, further comprising one or more computing devices in communication with the plurality of heating elements, the fill pump, the at least one feed mechanism, and the at least one fluid level sensor, the one or more computing devices comprising processor executable program code to cause one or more of the computing devices to:
receive fluid level sensor signals from the plurality of fluid level sensors;
determine the fluid levels within each trough of the subset based on the received fluid level sensor signals;
in response to determining that the fluid level of a respective trough of the subset is below a first threshold fluid level, transmit a signal to cause the fill valve to increase an amount of fluid provided to the respective trough; and
in response to determining that the fluid level is above a second threshold fluid level, transmit a signal to cause the fill valve to decrease the amount of fluid provided to the respective trough.

10. The system of claim 1, further comprising an actuator in physical communication with the at least one trough, the actuator positioned and configured to apply a force to the at least one trough to tilt the at least one trough.

11. The system of claim 1, wherein the at least one trough comprises one or more baffles disposed within the trough, each of the one or more baffles extending away from a bottom portion of the trough into an interior volume of the trough.

12. The system of claim 1, further comprising a heating element positioned to apply thermal energy to the at least one trough.

13. The system of claim 1, wherein the at least one lid further defines a port to receive food.

14. The system of claim 1, wherein the feeding mechanism comprises at least one of an augur, a blow feeder, or a broadcast feeder.

15. The system of claim 1, further comprising an air tube, a portion of the air tube positioned within the trough, the air tube connectable to a pressurized air source and defining a plurality of openings in the portion of the air tube positioned within the trough to provide pressurized air into the trough.

16. The system of claim 1, wherein the frame defines an enclosure having a plurality of walls, each wall comprising a plenum, each plenum having a plurality of openings to allow movement of air through the wall into the enclosure.

17. The system of claim 1, further comprising a robotic arm.

18. The system of claim 16, wherein at least one of the feeding mechanism or a camera is coupled to the robotic arm.

19. A method comprising:
dispensing a quantity of water into a trough of an automated insect rearing system, the automated insect rearing system comprising:
a frame,
at least one trough having a drain, the trough supported by the frame,
a drain valve coupled to the drain,
a lid couplable to the at least one trough to cover the trough, the lid defining at least one vent,
a fluid supply tube connected to a fluid supply, the fluid supply tube routed to provide fluid to the at least one trough,
a fill valve coupled to the fluid supply tube, the fill valve positioned and configured to regulate a flow of fluid into the at least one trough,
a fill pump coupled to the at least one fluid supply tube and coupled to the fluid supply to pump fluid from the fluid supply through the at least one supply tube,
at least one feeding mechanism to dispense food into the at least one trough, and
at least one fluid level sensor positioned to detect a fluid level within the at least one trough;
dispensing a quantity of insect larvae into the trough;
dispensing a quantity of food into the trough;
coupling a lid to the trough, the lid defining an opening;
activating, by the computing device, the fill pump to pump water from the fluid source into the fluid supply tube;
activating, by the computing device, an actuator to tilt the trough to distribute the food and larvae within the trough; and
activating, by the computing device, the actuator to return the trough to a substantially level position.

20. The method of claim 18, further comprising:
activating, by the computing device, a drain pump to pump water out of the trough at a predetermined flow rate;
determining, by the computing device based on one or more signals received from the fluid level sensor, a water level in the trough;
in response to determining that the fluid level is below a first threshold fluid level, transmit a signal to cause the fill valve to increase an amount of fluid provided to the at least one trough from the fluid supply tube; and
in response to determining that the fluid level is above a second threshold fluid level, transmit a signal to cause the fill valve to decrease the amount of fluid provided to the at least one trough from the fluid supply tube.

21. The method of claim 18, further comprising:
receiving, by the computing device, water quality sensor signals from a water quality sensor;
determine a water quality of the water within the trough based on the water quality sensor signals;
in response to determining that the water quality is below a first threshold water quality level, activating, by the computing device, a drain pump to pump water out of the trough at a predetermined flow rate.

22. The method of claim 18, further comprising:
determining, by the computing device, an elapsed time since a population of insect larvae was deposited within the at least one trough;
determining, by the computing device, a fluid replacement rate based on the elapsed time; and
transmitting, by the computing device, a signal to establish a flow rate of the drain pump based on the fluid replacement rate.

23. The method of claim 21, wherein the drain pump comprises a peristaltic pump.

24. The method of claim 21, further comprising, in response to determining, by the computing device, an unloading time based on the elapsed time:
transmitting an unload signal to the drain valve to open the drain valve; and
transmitting an actuator signal to the actuator to cause the actuator to tilt the at least one trough towards the drain.

25. The method of claim 18, further comprising pumping air into an air tube, a portion of the air tube positioned under the water within the trough, the portion of the air tube within the trough defining a plurality of openings to provide pressurized air into the water.

26. The system of claim 1, wherein the automated insect rearing system further comprises a heating element positioned to apply thermal energy to the at least one trough, and further comprising:
receiving, by a computing device, temperature sensor signals indicating a temperature of the water in the trough; and
adjusting, by the computing device, the heating element based on the temperature of the water and a predetermined water temperature.
